(19) United States
(12) Patent Application Publication
(10) Pub. No.: US 2026/0000548 A1
(43) Pub. Date: Jan. 1, 2026

(54) AIRWAY FOR A WOUND THERAPY DEVICE

(71) Applicant: CONVATEC LIMITED, Flintshire (GB)

(72) Inventor: Manjunath Penagondla, Warrington (GB)

(21) Appl. No.: 19/260,563

(22) Filed: Jul. 6, 2025

Related U.S. Application Data
(63) Continuation of application No. PCT/GB2024/050003, filed on Jan. 2, 2024.
(60) Provisional application No. 63/436,911, filed on Jan. 4, 2023.

(30) Foreign Application Priority Data
Feb. 15, 2023 (GB) 2302154.6

(51) Int. Cl. A61F 13/05; A61M 1/00

(52) U.S. Cl. CPC A61F 13/05; A61M 1/913; A61M 2207/00

(57) ABSTRACT

An airway for a pressure gradient wound therapy device comprising a spacer layer comprising a proximal end, an elongate middle portion and a distal end; a top layer constructed from a liquid impermeable material provided over the spacer layer; a bottom layer constructed from a liquid impermeable material provided below the spacer layer, wherein the top layer and the bottom layer substantially enclose the spacer layer; and a conduit comprising a proximal end and a distal end, wherein the proximal end of the conduit comprises at least a first elongate slot, wherein the first elongate slot forms at least a first split end and a second split end in the conduit, and further wherein at least part of the spacer layer is arranged in the elongate slot.

### TECHNICAL FIELD OF THE INVENTION

The present invention relates to an airway. In particular, the invention concerns an airway for a pressure gradient wound therapy device, and most particularly, but not exclusively, an airway for a negative pressure wound dressing.

### BACKGROUND TO THE INVENTION

Wound dressings are known and are generally suitable for treating a variety of wounds, including chronic and acute wound types, such as infected wounds, venous ulcers, diabetic ulcers, burns and surgical wounds.

Negative pressure has been used to treat a range of chronic and acute wounds. Negative pressure may facilitate wound healing through a number of mechanisms, including removal of excess exudate, reduction in periwound edema and increased perfusion. Combined with the physical forces exerted by the negative pressure which draw the wound edges together, this can result in improved wound outcomes. Existing wound dressing systems typically rely on an airway having a conduit to draw air away from the wound site via a pump.

One exemplary known airway is attached to Pico® dressings sold by Smith and Nephew®. This airway is a generally flat arrangement with a top layer, and a bottom layer which together define a channel, the channel with a spacer layer enclosed between the top and bottom layers.

Typically, the top and bottom layers are each a layer of flexible, transparent, liquid impermeable film, and are often formed of polyurethane. The spacer layer serves to keep the film layers separate to ensure that there is an open path for fluid to travel between the wound dressing and a source of negative pressure. For connection to a source of negative pressure, a short piece of transparent tubing extends from the end of the airway opposite to the wound dressing.

The spacer layer comprises an elongate portion (extending substantially the length of the channel) and a folded end which surrounds a first end of the tubing. The folded end is folded upward, toward the top layer, then toward itself so that the folded end is substantially parallel with the elongate portion. The fold comprises an aperture through which a first end of the conduit is inserted. The first end of the conduit is positioned between the folded portion of the spacer fabric and the elongate portion of the spacer fabric. Thus, the spacer layer maintains an open path for fluid from the wound dressing to the tubing. With a layer of spacer fabric on top and below, the film layer cannot close off the open first end of the tubing.

The arrangement of the prior art seemingly aims to reduce the possibility of an end of the conduit piercing either layer of film. However, the arrangement is flawed in that the conduit can easily be inserted too far through the aperture in the fold and extend beyond the folded portion of spacer fabric. This creates a pressure point between the conduit and the top film layer of which the conduit is then prone to piercing the top film layer. In this event, the patient may suffer from considerable discomfort should the conduit protrude into the patient's skin. However, this can also occur when the conduit extends beyond the folded portion of the spacer fabric and does not pierce the top film layer. Further, in the event that the conduit pierces the top film layer, the conduit would no longer be able to transmit a negative pressure to the wound site, therefore, adversely affecting healing and recovery of the wound.

Moreover, if the conduit is inserted too far through the aperture in the fold and extends beyond the folded portion of spacer fabric then, in use, the draw of air away from a wound site into the conduit would cause the top film layer to be drawn into the conduit, covering the opening to the conduit and, therefore, preventing the conduit transmitting a negative pressure to a wound site.

The arrangement of the prior art described above provides an airway which is relatively thin compared to, for example, wound dressings having solid parts (e.g., hard plastic tubing) extending from a backing layer of the dressing. However, it is considered that further improvements can be made.

It is an object of embodiments of the present invention to at least partially overcome or alleviate the above problems and/or to provide an improved airway for a wound dressing.

### SUMMARY OF THE INVENTION

In broad terms, the present invention concerns an airway for a pressure gradient wound therapy device. The airway may comprise a spacer layer. The airway may comprise a conduit. The conduit may comprise at least a first slot or cutaway. At least part of the spacer layer may be arranged in the slot or cutaway.

According to a broad aspect of the present invention, there is provided an airway for a pressure gradient wound therapy device comprising:
a spacer layer; and
a conduit comprising at least a first slot, wherein at least part of the spacer layer is arranged in the slot.

The spacer layer may comprise a proximal end. The spacer layer may comprise an elongate middle portion. The spacer layer may comprise a distal end. The airway may comprise a top layer. The top layer may be constructed from a liquid impermeable material. The top layer may be provided over the spacer layer. The airway may comprise a bottom layer. The bottom layer may be constructed from a liquid impermeable material. The bottom layer may be provided below the spacer layer. The top layer and the bottom layer may at least partially enclose the spacer layer. The top and bottom layer may substantially enclose the spacer layer. The conduit may comprise a proximal end. The conduit may comprise a distal end. The proximal end of the conduit may comprise at least the first slot. The slot may be elongate. The first elongate slot may form at least a first split end and a second split end of the conduit.

According to a first aspect of the invention there is provided an airway for a pressure gradient wound therapy device comprising a spacer layer comprising a proximal end, an elongate middle portion and a distal end; a top layer constructed from a liquid impermeable material provided over the spacer layer; a bottom layer constructed from a liquid impermeable material provided below the spacer layer, wherein the top layer and the bottom layer substantially enclose the spacer layer; and. a conduit comprising a proximal end and a distal end, wherein the proximal end of the conduit comprises at least a first elongate slot, wherein the first elongate slot forms at least a first split end and a second split end in the conduit, and further wherein at least part of the spacer layer is arranged in the elongate slot

Advantageously, the present invention provides an airway which is of reduced thickness compared to airways of the prior art. At its thickest part, the thickness of the airway of the present invention is approximately equal to the thickness of the conduit and the spacer layer. The spacer layer is not required to be folded or otherwise manipulated in order to accommodate the conduit. As such, the present invention provides an airway which is considerably more comfortable for a patient should the airway be rested or lent upon by the patient. Moreover, the airway is therefore less like to cause pressure ulcers and other complications which could otherwise result in significant distress and pain for the patient.

Further advantageously, the airway of the present invention is not prone to piercing a film layer should a film layer be positioned above and/or below the airway to enclose the airway. This is because at least part of the spacer layer is arranged in the first slot of the conduit, therefore, only a portion of the conduit is located either side of the spacer layer. Surprisingly, it has been found that because only a portion of an end of the conduit will be in contact with a film layer, this is not sufficient to easily pierce the film layer even in the event the airway is rested upon by a patient or during the normal handling of the airway in use.

Moreover, the present invention is far simpler to manufacture compared to airways of the prior art. The above-described airway of the prior art requires careful insertion of a conduit into an aperture created in a fold of a spacer layer. As previously stated, if the conduit is inserted too far, the conduit is prone to piercing the film enclosing the airway, therefore, careful manufacture of the airway of the prior art is critical, and even if made to high standards it is possible that in use the end of the conduit may poke out of the folded part. Comparatively, the present invention is simple to manufacture in that all that is required is a portion of the spacer fabric to be inserted into the slot of the conduit. As such, manufacture is easier compared to known airways, and pushing the conduit further into the airway will only serve to push the spacer layer into the slot, overcoming the issue with the prior art in terms of the conduit poking out of the folded part of the spacer layer in the prior art.

The top layer may be a film layer. The top layer may be provided over the conduit. In particular, the top layer may be provided over the conduit in the region of the slot.

The bottom layer may be a film layer. The bottom layer may be provided below the conduit. In particular the bottom layer may be provided below the conduit in the region of the slot.

By the top layer and the bottom layer substantially enclose the spacer layer it is meant that the top layer and bottom layer are joined together so as to at least partially enclose the spacer layer and form a channel with the spacer layer arranged therein. The top layer and bottom layer alone do not need to completely enclose the spacer layer. Indeed, there should at least be an opening at or near one end through which the conduit extends and an opening at or near the opposite end in communication with an opening in the wound dressing (in use). The spacer layer may be freely moveable within the channel formed by the top layer and the bottom layer.

For example, the top layer may cover the entire upper surface of the spacer layer and the bottom layer may cover the majority, or a substantial part of the underside of the spacer layer, and the top and bottom layers may be joined to enclose the spacer layer at the sides (preferably by an adhesive), but where the airway is connected to a wound dressing, it need not be enclosed by the top and bottom layers; in particular, the top layer may be bonded to the upper surface of the wound dressing, rather than the bottom layer to enclose the spacer layer in that region and provide a fluid-tight channel between the opening in the wound dressing and the conduit. The underside of the spacer layer may be unenclosed in the region of an aperture in the upper surface of the wound dressing. In this way there is fluid communication between interior of the wound dressing and the airway.

The top layer may be joined to an upper surface of a wound dressing by heat sealing, pressure sealing or by an adhesive.

The bottom layer may be joined to an upper surface of a wound dressing by heat sealing, pressure sealing or by an adhesive.

The conduit may be provided by a hollow tube, such as medical tubing. The hollow tube may be formed of polyurethane, thermoplastic polyurethane, thermoplastic elastomer, polyethylene, polypropylene, polyvinyl chloride, ethylene-vinyl acetate and a silicone material. The conduit allows the passage of fluid (i.e., a gas or a liquid) along its length or at least along a substantial portion of its length.

The first split end may be provided above the proximal end of the spacer layer.

The second split end may be provided below the proximal end of the spacer layer.

Due to the arrangement of the spacer layer between the first split end and the second split end, the first split end may exert a force downwardly onto the spacer layer, and the second split end may exert a force upwardly onto the spacer layer. Such an arrangement reliably holds the spacer layer between the first split end and the second split end. Thus, displacing the spacer layer from the slot, i.e., between the first split end and the second split end, would require greater force than if the conduit comprised a single split end and the spacer layer was arranged upon or below such a split end. As such, the at least part of the spacer layer may be reliably held within the slot without the use of a sealing means (for example heat sealing or an adhesive) between the first split end and the spacer layer, and the second split end and the spacer layer.

Each split end may taper towards its tip. A tapered split end may provide each split end with a substantially elongate elliptical opening. In some embodiments, the taper of the first split end and/or second split end may be so gradual as to allow the respective split end to sit almost flat against a portion of an upper surface of the spacer layer or a portion of a lower surface (i.e., underside) of the spacer layer, respectively.

Advantageously, in embodiments comprising a first split end and/or second split end which tapers towards its tip, the or each split end may comprise an outer surface which becomes flatter towards its end and, being thinner, is more flexible towards its end, and therefore does not comprise a hard/sharp end point which the top layer or bottom layer could be pressed against and which may, albeit unlikely, cause the conduit to pierce the respective film layer. As such, beneficially, in embodiments where each split end tapers, the possibility that the conduit will pierce the top and/or bottom film layers is further reduced.

The first slot may be formed as a diametrical slot at the proximal end of the conduit, in particular into the terminus of the proximal end of the conduit.

The slot may be formed as a slit, with no material removed from the proximal end. As such, the split ends may meet at their bases. Alternatively, the slot may be formed by removing material, such that the split ends are separated by a gap at their bases.

The gap may have a distance between the lowermost part of the first split end and the uppermost part of the second split end of about 0.2 mm, about 0.3 mm, about 0.4 mm, about 0.5 mm, about 0.6 mm, about 0.7 mm, about 0.8 mm, about 0.9 mm, about 1.0 mm, about 1.10 mm, about 1.20 mm, about 1.30 mm, about 1.40 mm, about 1.50 mm, about 1.60 mm, about 1.70 mm, about 1.80 mm, about 1.90 mm, about 2.00 mm, about 2.10 mm, about 2.20 mm, about 2.30 mm, about 2.40 mm, about 2.50 mm, about 2.60 mm, about 2.70 mm, about 2.80 mm, about 2.90 mm, or about 3.00 mm.

The gap may have a distance between the lowermost part of the first split end and the uppermost part of the second split end of at least about 0.2 mm, about 0.3 mm, about 0.4 mm, about 0.5 mm, about 0.6 mm, about 0.7 mm, about 0.8 mm, about 0.9 mm, about 1.0 mm, about 1.10 mm, about 1.20 mm, about 1.30 mm, about 1.40 mm, about 1.50 mm, about 1.60 mm, about 1.70 mm, about 1.80 mm, about 1.90 mm, about 2.00 mm, about 2.10 mm, about 2.20 mm, about 2.30 mm, about 2.40 mm, about 2.50 mm, about 2.60 mm, about 2.70 mm, about 2.80 mm, about 2.90 mm, or at least about 3.00 mm.

The gap may have a distance between the lowermost part of the first split end and the uppermost part of the second split end of no more than about 0.2 mm, about 0.3 mm, about 0.4 mm, about 0.5 mm, about 0.6 mm, about 0.7 mm, about 0.8 mm, about 0.9 mm, about 1.0 mm, about 1.10 mm, about 1.20 mm, about 1.30 mm, about 1.40 mm, about 1.50 mm, about 1.60 mm, about 1.70 mm, about 1.80 mm, about 1.90 mm, about 2.00 mm, about 2.10 mm, about 2.20 mm, about 2.30 mm, about 2.40 mm, about 2.50 mm, about 2.60 mm, about 2.70 mm, about 2.80 mm, about 2.90 mm, or no more than about 3.00 mm.

The conduit may comprise a straight cut. The proximal end of the conduit may comprise the straight cut. The straight cut may extend to the terminus of the proximal end of the conduit. Advantageously, this provides a large surface area of the proximal end of the conduit toward which the spacer layer can be guided. Thus, this facilitates insertion of the spacer layer into the slot during manufacture of the airway. Moreover, forming the proximal end by a straight cut is an efficient and simple manufacturing technique which can facilitate manufacture of the airway. In manufacture of the airway, the conduit may be pinched at the base of the first slot to separate the first split end and the second split end to temporarily enlarge the first slot. This facilitates insertion of the spacer layer into the slot. The first slot may extend into the conduit from the straight cut. The slot may extend from the straight cut into the conduit. The slot may extend perpendicular to the straight cut.

The conduit may comprise a notched cut. The proximal end of the conduit may comprise the notched cut. The notched cut may comprise an apex. The slot may extend into the conduit from the notched cut, in particular from the apex of the notched cut. Each split end may taper outwardly from the centre of the conduit toward the proximal end of the conduit. In particular, each split end may taper outwardly from the apex of the notched cut toward the proximal end of the conduit. Advantageously, the notched cut facilitates insertion of the spacer layer into the first slot during manufacture of the airway as the tapered split ends guide the spacer layer into the first slot. In manufacture of the airway, the conduit may be pinched at the base of the first slot to separate the first split end and the second split end to temporarily enlarge the first slot. The notched cut may be a notched triangular cut. The first slot may extend into the conduit from the notched cut, e.g. from the apex of the triangular notched cut.

The conduit may comprise a second slot. The second slot may be perpendicular to the first slot. The second slot may be formed in the same way as the first slot, i.e., the second slot may be formed as a diametrical slot at the proximal end of the conduit, in particular into the terminus of the proximal end of the conduit. The proximal end of the conduit may comprise the second slot. The second slot may be an elongate slot. The second slot may be formed by a straight cut or a notched cut, as described above in relation to the first slot. Likewise, the second slot may be formed as a slit or alternatively, by removing material, such that the split ends are separated by a gap at their bases.

The second slot may form a third split end and a fourth split end. The third split end may be of equal, or substantially equal, dimensions to the first, second and/or fourth split end. The fourth split end may be of equal, or substantially equal, dimensions to the first, second and/or third split end. Each split end may taper towards its tip. Advantageously, this means that each split end comprises an outer surface which becomes flatter towards its end and, being thinner, is more flexible towards its end, and therefore does not comprise a hard/sharp end point which the top layer or bottom layer could be pressed against and which may, albeit unlikely, cause the conduit to pierce the respective film layer. As such, beneficially, in embodiments where each split end tapers, the possibility that the conduit will pierce the top and/or bottom film layers is further reduced.

The proximal end of the spacer layer may be arranged between two pairs of split ends. Advantageously, this arrangement provides for a greater grip of the spacer layer between the split ends. As such, the spacer layer may be held in place between the split ends by friction fit. Thus, this arrangement in particular does not require the use of an adhesive or weld to adhere the spacer layer to the split ends. As such, this arrangement is simple and cost effective to manufacture. Moreover, greater splitting makes for thinner, more flexible ends, which are less likely to pierce the top or bottom layer and may be more comfortable if the user lies on the airway.

The proximal end of the spacer layer may be arranged between a first pair of split ends comprising the first and third split ends, and a second pair of split ends comprising the second and fourth split ends.

The proximal end of the spacer layer may be arranged such that a first pair of split ends is provided above the proximal end of the spacer layer. The first pair of split ends may comprise the first and third split ends. The first pair of split ends may exert a pressure downwardly onto the spacer layer.

The proximal end of the spacer layer may be arranged such that a second pair of split ends is provided below the proximal end of the spacer layer. The second pair of split ends may comprise the second and fourth split ends. The second pair of split ends may exert a pressure upwardly onto the spacer layer.

The first slot may have a length of about 5 mm, about 6 mm, about 7 mm, about 8 mm, about 9 mm, about 10 mm, about 12 mm, about 14 mm, about 15 mm, about 16 mm, about 18 mm, about 20 mm, about 22 mm, about 24 mm, about 25 mm, about 26 mm, about 28 mm, or about 30 mm.

The first slot may have a length of at least about 5 mm, about 6 mm, about 7 mm, about 8 mm, about 9 mm, about 10 mm, about 12 mm, about 14 mm, about 15 mm, about 16 mm, about 18 mm, about 20 mm, about 22 mm, about 24 mm, about 25 mm, about 26 mm, about 28 mm, or at least about 30 mm.

The first slot may have a length of no more than about 5 mm, about 6 mm, about 7 mm, about 8 mm, about 9 mm, about 10 mm, about 12 mm, about 14 mm, about 15 mm, about 16 mm, about 18 mm, about 20 mm, about 22 mm, about 24 mm, about 25 mm, about 26 mm, about 28 mm, or no more than about 30 mm.

The first slot may have a length of from about 5 mm to about 30 mm, from about 5 mm to about 25 mm, from about 5 mm to about 20 mm, from about 5 mm to about 15 mm, or from about 5 mm to about 10 mm.

The first slot may have a length of from about 10 mm to about 30 mm, from about 10 mm to about 25 mm, from about 10 mm to about 20 mm, or from about 10 mm to about 15 mm.

The first slot may have a length of from about 15 mm to about 30 mm, from about 15 mm to about 25 mm, or from about 15 mm to about 20 mm.

The first slot may have a length of from about 20 mm to about 30 mm, or from about 20 mm to about 25 mm.

The first slot may have a length of from about 25 mm to about 30 mm.

Advantageously, this allows the spacer layer to be held within the slot, between the first and second split ends, by friction. Thus, the airway can be manufactured without using an adhesive to adhere the conduit to the spacer layer. Further advantageously, the length of the first slot allows the first slot to hold spacer layers of different thicknesses.

The second slot may have a length of about 5 mm, about 6 mm, about 7 mm, about 8 mm, about 9 mm, about 10 mm, about 12 mm, about 14 mm, about 15 mm, about 16 mm, about 18 mm, about 20 mm, about 22 mm, about 24 mm, about 25 mm, about 26 mm, about 28 mm, or about 30 mm.

The second slot may have a length of at least about 5 mm, about 6 mm, about 7 mm, about 8 mm, about 9 mm, about 10 mm, about 12 mm, about 14 mm, about 15 mm, about 16 mm, about 18 mm, about 20 mm, about 22 mm, about 24 mm, about 25 mm, about 26 mm, about 28 mm, or at least about 30 mm.

The second slot may have a length of no more than about 5 mm, about 6 mm, about 7 mm, about 8 mm, about 9 mm, about 10 mm, about 12 mm, about 14 mm, about 15 mm, about 16 mm, about 18 mm, about 20 mm, about 22 mm, about 24 mm, about 25 mm, about 26 mm, about 28 mm, or no more than about 30 mm.

The second slot may have a length of from about 5 mm to about 30 mm, from about 5 mm to about 25 mm, from about 5 mm to about 20 mm, from about 5 mm to about 15 mm, or from about 5 mm to about 10 mm.

The second slot may have a length of from about 10 mm to about 30 mm, from about 10 mm to about 25 mm, from about 10 mm to about 20 mm, or from about 10 mm to about 15 mm.

The second slot may have a length of from about 15 mm to about 30 mm, from about 15 mm to about 25 mm, or from about 15 mm to about 20 mm.

The second slot may have a length of from about 20 mm to about 30 mm, or from about 20 mm to about 25 mm.

The second slot may have a length of from about 25 mm to about 30 mm.

The first slot and the second slot may have the same length or may have different lengths. For example, the first slot may have a greater length than the second slot, or the second slot may have a greater length than the first slot.

The first slot may have a length equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the length of the conduit.

The first slot may have a length equal to no more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or no more than about 95% of the length of the conduit.

The first slot may have a length equal to at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or at least about 95% of the length of the conduit.

The first slot may have a length of from about 5% to about 95%, from about 5% to about 90%, from about 5% to about 85%, from about 5% to about 80%, from about 5% to about 75%, from about 5% to about 70%, from about 5% to about 65%, from about 5% to about 60%, from about 5% to about 55%, from about 5% to about 50%, from about 5% to about 45%, from about 5% to about 40%, from about 5% to about 35%, from about 5% to about 30%, from about 5% to about 25%, from about 5% to about 20%, from about 5% to about 15%, or from about 5% to about 10% of the length of the conduit.

The first slot may have a length of from about 10% to about 95%, from about 10% to about 90%, from about 10% to about 85%, from about 10% to about 80%, from about 10% to about 75%, from about 10% to about 70%, from about 10% to about 65%, from about 10% to about 60%, from about 10% to about 55%, from about 10% to about 50%, from about 10% to about 45%, from about 10% to about 40%, from about 10% to about 35%, from about 10% to about 30%, from about 10% to about 25%, from about 10% to about 20%, or from about 10% to about 15%, of the length of the conduit.

The first slot may have a length of from about 15% to about 95%, from about 15% to about 90%, from about 15% to about 85%, from about 15% to about 80%, from about 15% to about 75%, from about 15% to about 70%, from about 15% to about 65%, from about 15% to about 60%, from about 15% to about 55%, from about 15% to about 50%, from about 15% to about 45%, from about 15% to about 40%, from about 15% to about 35%, from about 15% to about 30%, from about 15% to about 25%, or from about 15% to about 20%, of the length of the conduit.

The first slot may have a length of from about 20% to about 95%, from about 20% to about 90%, from about 20% to about 85%, from about 20% to about 80%, from about 20% to about 75%, from about 20% to about 70%, from about 20% to about 65%, from about 20% to about 60%, from about 20% to about 55%, from about 20% to about 50%, from about 20% to about 45%, from about 20% to about 40%, from about 20% to about 35%, from about 20% to about 30%, or from about 20% to about 25%, of the length of the conduit.

The first slot may have a length of from about 25% to about 95%, from about 25% to about 90%, from about 25% to about 85%, from about 25% to about 80%, from about 25% to about 75%, from about 25% to about 70%, from about 25% to about 65%, from about 25% to about 60%, from about 25% to about 55%, from about 25% to about 50%, from about 25% to about 45%, from about 25% to about 40%, from about 25% to about 35%, or from about 25% to about 30%, of the length of the conduit.

The first slot may have a length of from about 30% to about 95%, from about 30% to about 90%, from about 30% to about 85%, from about 30% to about 80%, from about 30% to about 75%, from about 30% to about 70%, from about 30% to about 65%, from about 30% to about 60%, from about 30% to about 55%, from about 30% to about 50%, from about 30% to about 45%, from about 30% to about 40%, or from about 30% to about 35%, of the length of the conduit.

The first slot may have a length of from about 35% to about 95%, from about 35% to about 90%, from about 35% to about 85%, from about 35% to about 80%, from about 35% to about 75%, from about 35% to about 70%, from about 35% to about 65%, from about 35% to about 60%, from about 35% to about 55%, from about 35% to about 50%, from about 35% to about 45%, or from about 35% to about 40%, of the length of the conduit.

The first slot may have a length of from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, or from about 40% to about 45%, of the length of the conduit.

The first slot may have a length of from about 45% to about 95%, from about 45% to about 90%, from about 45% to about 85%, from about 45% to about 80%, from about 45% to about 75%, from about 45% to about 70%, from about 45% to about 65%, from about 45% to about 60%, from about 45% to about 55%, or from about 45% to about 50%, of the length of the conduit.

The first slot may have a length of from about 50% to about 95%, from about 50% to about 90%, from about 50% to about 85%, from about 50% to about 80%, from about 50% to about 75%, from about 50% to about 70%, from about 50% to about 65%, from about 50% to about 60%, or from about 50% to about 55%, of the length of the conduit.

The first slot may have a length of from about 55% to about 95%, from about 55% to about 90%, from about 55% to about 85%, from about 55% to about 80%, from about 55% to about 75%, from about 55% to about 70%, from about 55% to about 65%, or from about 55% to about 60%, of the length of the conduit.

The first slot may have a length of from about 60% to about 95%, from about 60% to about 90%, from about 60% to about 85%, from about 60% to about 80%, from about 60% to about 75%, from about 60% to about 70%, or from about 60% to about 65%, of the length of the conduit.

The first slot may have a length of from about 65% to about 95%, from about 65% to about 90%, from about 65% to about 85%, from about 65% to about 80%, from about 65% to about 75%, or from about 65% to about 70%, of the length of the conduit.

The first slot may have a length of from about 70% to about 95%, from about 70% to about 90%, from about 70% to about 85%, from about 70% to about 80%, or from about 70% to about 75%, of the length of the conduit.

The first slot may have a length of from about 75% to about 95%, from about 75% to about 90%, from about 75% to about 85%, or from about 75% to about 80%, the length of the conduit.

The first slot may have a length of from about 80% to about 95%, from about 80% to about 90%, or from about 80% to about 85%, the length of the conduit.

The first slot may have a length of from about 85% to about 95%, or from about 85% to about 90%, the length of the conduit.

The first slot may have a length of from about 90% to about 95%, the length of the conduit.

The second slot may have a length equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the length of the conduit.

The second slot may have a length equal to no more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or no more than about 95% of the length of the conduit.

The second slot may have a length equal to at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or at least about 95% of the length of the conduit.

The second slot may have a length of from about 5% to about 95%, from about 5% to about 90%, from about 5% to about 85%, from about 5% to about 80%, from about 5% to about 75%, from about 5% to about 70%, from about 5% to about 65%, from about 5% to about 60%, from about 5% to about 55%, from about 5% to about 50%, from about 5% to about 45%, from about 5% to about 40%, from about 5% to about 35%, from about 5% to about 30%, from about 5% to about 25%, from about 5% to about 20%, from about 5% to about 15%, or from about 5% to about 10% of the length of the conduit.

The second slot may have a length of from about 10% to about 95%, from about 10% to about 90%, from about 10% to about 85%, from about 10% to about 80%, from about 10% to about 75%, from about 10% to about 70%, from about 10% to about 65%, from about 10% to about 60%, from about 10% to about 55%, from about 10% to about 50%, from about 10% to about 45%, from about 10% to about 40%, from about 10% to about 35%, from about 10% to about 30%, from about 10% to about 25%, from about 10% to about 20%, or from about 10% to about 15%, of the length of the conduit.

The second slot may have a length of from about 15% to about 95%, from about 15% to about 90%, from about 15% to about 85%, from about 15% to about 80%, from about 15% to about 75%, from about 15% to about 70%, from about 15% to about 65%, from about 15% to about 60%, from about 15% to about 55%, from about 15% to about 50%, from about 15% to about 45%, from about 15% to about 40%, from about 15% to about 35%, from about 15% to about 30%, from about 15% to about 25%, or from about 15% to about 20%, of the length of the conduit.

The second slot may have a length of from about 20% to about 95%, from about 20% to about 90%, from about 20% to about 85%, from about 20% to about 80%, from about 20% to about 75%, from about 20% to about 70%, from about 20% to about 65%, from about 20% to about 60%, from about 20% to about 55%, from about 20% to about 50%, from about 20% to about 45%, from about 20% to about 40%, from about 20% to about 35%, from about 20% to about 30%, or from about 20% to about 25%, of the length of the conduit.

The second slot may have a length of from about 25% to about 95%, from about 25% to about 90%, from about 25% to about 85%, from about 25% to about 80%, from about 25% to about 75%, from about 25% to about 70%, from about 25% to about 65%, from about 25% to about 60%, from about 25% to about 55%, from about 25% to about 50%, from about 25% to about 45%, from about 25% to about 40%, from about 25% to about 35%, or from about 25% to about 30%, of the length of the conduit.

The second slot may have a length of from about 30% to about 95%, from about 30% to about 90%, from about 30% to about 85%, from about 30% to about 80%, from about 30% to about 75%, from about 30% to about 70%, from about 30% to about 65%, from about 30% to about 60%, from about 30% to about 55%, from about 30% to about 50%, from about 30% to about 45%, from about 30% to about 40%, or from about 30% to about 35%, of the length of the conduit.

The second slot may have a length of from about 35% to about 95%, from about 35% to about 90%, from about 35% to about 85%, from about 35% to about 80%, from about 35% to about 75%, from about 35% to about 70%, from about 35% to about 65%, from about 35% to about 60%, from about 35% to about 55%, from about 35% to about 50%, from about 35% to about 45%, or from about 35% to about 40%, of the length of the conduit.

The second slot may have a length of from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, or from about 40% to about 45%, of the length of the conduit.

The second slot may have a length of from about 45% to about 95%, from about 45% to about 90%, from about 45% to about 85%, from about 45% to about 80%, from about 45% to about 75%, from about 45% to about 70%, from about 45% to about 65%, from about 45% to about 60%, from about 45% to about 55%, or from about 45% to about 50%, of the length of the conduit.

The second slot may have a length of from about 50% to about 95%, from about 50% to about 90%, from about 50% to about 85%, from about 50% to about 80%, from about 50% to about 75%, from about 50% to about 70%, from about 50% to about 65%, from about 50% to about 60%, or from about 50% to about 55%, of the length of the conduit.

The second slot may have a length of from about 55% to about 95%, from about 55% to about 90%, from about 55% to about 85%, from about 55% to about 80%, from about 55% to about 75%, from about 55% to about 70%, from about 55% to about 65%, or from about 55% to about 60%, of the length of the conduit.

The second slot may have a length of from about 60% to about 95%, from about 60% to about 90%, from about 60% to about 85%, from about 60% to about 80%, from about 60% to about 75%, from about 60% to about 70%, or from about 60% to about 65%, of the length of the conduit.

The second slot may have a length of from about 65% to about 95%, from about 65% to about 90%, from about 65% to about 85%, from about 65% to about 80%, from about 65% to about 75%, or from about 65% to about 70%, of the length of the conduit.

The second slot may have a length of from about 70% to about 95%, from about 70% to about 90%, from about 70% to about 85%, from about 70% to about 80%, or from about 70% to about 75%, of the length of the conduit.

The second slot may have a length of from about 75% to about 95%, from about 75% to about 90%, from about 75% to about 85%, or from about 75% to about 80%, the length of the conduit.

The second slot may have a length of from about 80% to about 95%, from about 80% to about 90%, or from about 80% to about 85%, the length of the conduit.

The second slot may have a length of from about 85% to about 95%, or from about 85% to about 90%, the length of the conduit.

The second slot may have a length of from about 90% to about 95%, the length of the conduit.

Advantageously, the length of the first and/or second slots, as described above, allows the spacer layer to be held within the slot, between the first and second split ends, by friction or with the use of a relatively small amount of an adhesive or by relatively minor sealing means, for example heat sealing or pressure sealing. Thus, in some embodiments, the airway can be manufactured without using an adhesive, or with using only a small amount of adhesive, to adhere the conduit to the spacer layer. Further advantageously, the length of the first slot allows the first slot to hold spacer layers of different thicknesses. Thus, beneficially, in manufacture of the airway, a conduit may be manufactured and it may find applicability with various types of spacer fabric, for example spacer layers formed of a range of materials (for example having different densities and/or formed of varying thicknesses), depending on the end use of the airway.

The spacer layer may be planar or at least substantially planar.

The spacer layer may be planar, or at least substantially planar at its portion which is arranged in the elongate slot.

The spacer layer may not be folded, or at least not substantially folded, at its portion which is arranged in the elongate slot.

Advantageously, in embodiments comprising a spacer layer which may planar or at least substantially planar, and in embodiments comprising a spacer layer which may be planar, or at least substantially planar at its portion which is arranged in the elongate slot, and in embodiments comprising a spacer layer which may not be folded, or at least not substantially folded, at its portion which is arranged in the elongate slot, the airway provides a more planar (i.e., flatter) arrangement which is considerably more comfortable for a patient should the airway be rested or lent upon by the patient. Moreover, the airway is therefore less like to cause pressure ulcers and other complications which could otherwise result in significant distress and pain for the patient.

The spacer layer is well known in the art, and can be derived from a number of materials. The materials are typically capable of forming filaments. As such, the spacer layer may comprise a plurality of filaments between opposing layers, for example opposing fabric layers.

The spacer layer may be a braided or a knitted material.

Typically, the spacer layer may be formed from a polyester, nylon, or polyurethane. Polyester microfilaments are commonly used examples of a spacer layer.

The knitted material may be, for example, a knitted mesh, for example a nylon knitted mesh. The knitted material may be a 3D knitted material.

The braided material may be, for example, a braided mesh, for example a nylon braided mesh. The braided material may be a 3D braided material.

The spacer layer may be a foam, for example a polyurethane foam.

The spacer layer may be a spacer fabric, for example a 3D spacer fabric. The spacer fabric may be a polyester spacer fabric. The spacer fabric may comprise a microfilament yarn between two fabric layers. The yarn and/or one or each of the fabric layers may be formed of polyester.

The spacer layer may be a porous material.

The spacer layer may be a non-woven material.

Advantageously, each of these materials are cheap and readily available. Moreover, many of these materials are recyclable and reusable, and can be formed from recycled materials.

Each split end may extend across the proximal end of the spacer layer a length equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the length of the spacer layer.

Each split end may extend across the proximal end of the spacer layer a length equal to at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or at least about 95% of the length of the spacer layer.

Each split end may extend across the proximal end of the spacer layer a length of no more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or no more than about 95% of the length of the spacer layer.

Advantageously, the split ends may extend across the proximal end of the spacer layer to a relatively short length compared to the overall length of the spacer layer, for example the split ends may extend across the proximal end of the spacer layer a length of no more than 10% of the length of the spacer layer. Beneficially, in embodiments where the split ends are adhered to the spacer layer, a relatively small amount of adhesive is required. Moreover, despite the split ends extending across the proximal end of the spacer layer to a relatively short length, the spacer layer can be securely held between the split ends without using an adhesive, i.e., the spacer layer may be held between the split ends by friction.

Each split end may have a different length. In embodiments where there are more than two split ends, at least one split end may have a different length to that of the other split ends. For example, in embodiments comprising four split ends, all four split ends may have the same or different lengths, or three split ends may have the same length and one split end may have a different length, or two split ends may have the same length and two split ends may have different or the same lengths.

In embodiments comprising a first split end and a second split end, the first split end may have a shorter length than the second split end. This may facilitate insertion of the spacer layer between the split ends as the slot may be opened by gently pressing the spacer layer onto the second split end to move the two split ends away from each other. The spacer layer may then be easily inserted into the slot.

In embodiments comprising four split ends, the two split ends arranged above the proximal end of the spacer layer may each have a shorter length than that of the two split ends arranged below the proximal end of the spacer layer. This may facilitate insertion of the spacer layer between the two split ends arranged above the proximal end of the spacer layer and the two split ends arranged below the proximal end of the spacer layer, as the slot may be opened by gently pressing the spacer layer onto the two split ends arranged below the proximal end of the spacer layer to move the pairs of split ends away from each other.

The spacer layer may be held between the first and second split ends by friction. Advantageously, this means that the airway does not require the use of an adhesive or weld to adhere the spacer layer to the one or more split ends. As such, this arrangement is simple and cost effective to manufacture.

The spacer layer may be held between the first and second split ends by heat lamination, pressure lamination, heat and pressure lamination, welding, for example a heat weld, or an adhesive. Advantageously, the present invention affords a physical ‘grip’ upon the spacer layer when arranged between the first split end and the second split end. Thus, beneficially, the aforementioned heat lamination, pressure lamination, heat and pressure lamination, welding, for example a heat weld, or an adhesive, may not be required to hold the spacer layer between the first and second split ends, or at least, for example in embodiments comprising an adhesive to hold the spacer layer between the split ends, a relatively small quantity of adhesive only may be required.

The width of each of the proximal end, elongate middle portion and distal end of the spacer layer may be equal, or at least substantially equal. The spacer layer may comprise an enlarged proximal end, elongate middle portion and/or distal end. The enlarged proximal end, elongate middle portion and/or distal end may comprise a pair of wings or a circular portion of spacer fabric having a total width greater than the width of the spacer layer where the wings or circular portion are not present. The enlarged portion may allow the spacer layer to accommodate an adhesive, for example a double-sided adhesive, for adhering the spacer layer to a top or bottom layer, as desired.

The spacer layer may have a thickness of between about 0.50 mm and about 4.50 mm, about 0.60 mm and about 4.40 mm, about 0.65 mm and about 4.30 mm, about 0.70 mm and about 4.20 mm, about 0.75 mm and about 4.10 mm, about 0.80 mm and about 4.00 mm, about 0.85 mm and about 3.90 mm, about 0.90 mm and about 3.80 mm, about 0.95 mm and about 3.70 mm, about 1.00 mm and about 3.60 mm, about 1.05 mm and about 3.50 mm, about 1.10 mm and about 3.40 mm, about 1.15 mm and about 3.30 mm, about 1.20 mm and about 3.20 mm, about 1.25 mm and about 3.10 mm, about 1.30 mm and about 3.00 mm, about 1.35 mm and about 2.90 mm, about 1.40 mm and about 2.80 mm, about 1.45 mm and about 2.70 mm, about 1.50 mm and about 2.60 mm, about 1.55 mm and about 2.50 mm, about 1.60 mm and about 2.40 mm, about 1.65 mm and about 2.30 mm, about 1.70 mm and about 2.20 mm, about 1.75 mm and about 2.10 mm, about 1.80 mm and about 2.00 mm, or about 1.90 mm.

The spacer layer may have a thickness of about 0.50 mm, about 0.55 mm, about 0.60 mm, about 0.65 mm, about 0.70 mm, about 0.75 mm, about 0.80 mm, about 0.85 mm, about 0.90 mm, about 0.95 mm, about 1.00 mm, about 1.05 mm, about 1.10 mm, about 1.15 mm, about 1.20 mm, about 1.25 mm, about 1.30 mm, about 1.35 mm, about 1.40 mm, about 1.45 mm, about 1.50 mm, about 1.55 mm, about 1.60 mm, about 1.65 mm, about 1.70 mm, about 1.75 mm, about 1.80 mm, about 1.85 mm, about 1.90 mm, about 1.95 mm, about 2.00 mm, about 2.05 mm, about 2.10 mm, about 2.15 mm, about 2.20 mm, about 2.25 mm, about 2.30 mm, about 2.35 mm, about 2.40 mm, about 2.45 mm, about 2.50 mm, about 2.55 mm, about 2.60 mm, about 2.65 mm, about 2.70 mm, about 2.75 mm, about 2.80 mm, about 2.85 mm, about 2.90 mm, about 2.95 mm, about 3.00 mm, about 3.10 mm, about 3.20 mm, about 3.30 mm, about 3.40 mm, about 3.50 mm, about 3.60 mm, about 3.70 mm, about 3.80 mm, about 3.90 mm, or about 4.00 mm.

The spacer layer may have a thickness of at least about 0.50 mm, about 0.55 mm, about 0.60 mm, about 0.65 mm, about 0.70 mm, about 0.75 mm, about 0.80 mm, about 0.85 mm, about 0.90 mm, about 0.95 mm, about 1.00 mm, about 1.05 mm, about 1.10 mm, about 1.15 mm, about 1.20 mm, about 1.25 mm, about 1.30 mm, about 1.35 mm, about 1.40 mm, about 1.45 mm, about 1.50 mm, about 1.55 mm, about 1.60 mm, about 1.65 mm, about 1.70 mm, about 1.75 mm, about 1.80 mm, about 1.85 mm, about 1.90 mm, about 1.95 mm, about 2.00 mm, about 2.05 mm, about 2.10 mm, about 2.15 mm, about 2.20 mm, about 2.25 mm, about 2.30 mm, about 2.35 mm, about 2.40 mm, about 2.45 mm, about 2.50 mm, about 2.55 mm, about 2.60 mm, about 2.65 mm, about 2.70 mm, about 2.75 mm, about 2.80 mm, about 2.85 mm, about 2.90 mm, about 2.95 mm, about 3.00 mm, about 3.10 mm, about 3.20 mm, about 3.30 mm, about 3.40 mm, about 3.50 mm, about 3.60 mm, about 3.70 mm, about 3.80 mm, about 3.90 mm, or at least about 4.00 mm.

The spacer layer may have a thickness of no more than about 0.50 mm, about 0.55 mm, about 0.60 mm, about 0.65 mm, about 0.70 mm, about 0.75 mm, about 0.80 mm, about 0.85 mm, about 0.90 mm, about 0.95 mm, about 1.00 mm, about 1.05 mm, about 1.10 mm, about 1.15 mm, about 1.20 mm, about 1.25 mm, about 1.30 mm, about 1.35 mm, about 1.40 mm, about 1.45 mm, about 1.50 mm, about 1.55 mm, about 1.60 mm, about 1.65 mm, about 1.70 mm, about 1.75 mm, about 1.80 mm, about 1.85 mm, about 1.90 mm, about 1.95 mm, about 2.00 mm, about 2.05 mm, about 2.10 mm, about 2.15 mm, about 2.20 mm, about 2.25 mm, about 2.30 mm, about 2.35 mm, about 2.40 mm, about 2.45 mm, about 2.50 mm, about 2.55 mm, about 2.60 mm, about 2.65 mm, about 2.70 mm, about 2.75 mm, about 2.80 mm, about 2.85 mm, about 2.90 mm, about 2.95 mm, about 3.00 mm, about 3.10 mm, about 3.20 mm, about 3.30 mm, about 3.40 mm, about 3.50 mm, about 3.60 mm, about 3.70 mm, about 3.80 mm, about 3.90 mm, or no more than about 4.00 mm.

The spacer layer may be any shape, such as, but not limited to, circular, square, triangular or rectangular.

The spacer layer may be adhered to at least one of the top and bottom layers. The spacer layer may be adhered to at least one of the top and bottom layers by heat lamination, pressure lamination, heat and pressure lamination, welding, for example a heat weld, or an adhesive, for example a pressure-sensitive adhesive.

At least one of the top layer and the bottom layer, and preferably of the top layer and the bottom layer, may be formed of polyurethane, thermoplastic polyurethane, thermoplastic elastomer, polyethylene, polypropylene, polyvinyl chloride, ethylene-vinyl acetate or a silicone material.

The top layer and the bottom layer may be formed of the same material.

At least one of the top layer and the bottom layer and preferably of the top layer and the bottom layer, may be waterproof, gas impermeable and/or moisture vapour permeable. Advantageously, a liquid and gas impermeable, but moisture vapour permeable top and/or bottom layer ensures that gas does not escape from the airway, reducing the vacuum; that liquid does not escape from the airway (e.g. on route to a cannister, if the NPWT system is one including a cannister), but allows moisture vapour to escape, reducing the quantity of moisture that must be held in the cannister.

The top layer may be adhered to the bottom layer around its periphery to form an elongate channel holding the spacer layer therein. The top layer may be adhered to the bottom layer by heat lamination, pressure lamination, heat and pressure lamination, welding, for example a heat weld, or an adhesive, for example a pressure-sensitive adhesive.

The top layer and the bottom layer may enclose, the proximal end of the spacer layer. The top layer and the bottom layer may enclose the proximal end and the elongate middle portion of the spacer layer. The top layer may enclose the proximal end, elongate middle portion and the distal end of the spacer layer and the bottom layer may enclose the proximal end and at least part, for example a major part, of the elongate middle portion. The bottom layer may not enclose the distal end of the spacer layer. An aperture may be provided in the bottom layer for connection to an opening in a wound dressing. The top layer and the bottom layer may enclose the elongate middle portion of the conduit. The top layer and the bottom layer may enclose the distal end of the conduit.

The airway may be suitable and intended for use with a portless negative pressure wound dressing.

By ‘portless’, we mean a negative pressure wound dressing which does not comprise a port. In the absence of an airway, a port is used to connect a source of negative pressure (e.g. via medical tubing) to a wound dressing.

According to a second aspect of the invention, there is provided the use of an airway according to the first aspect with a portless negative pressure wound dressing (optionally including any optional features).

According to a third aspect of the invention, there is provided a negative pressure wound dressing comprising the airway according to the first aspect (optionally including any optional features). The airway may be attached to a backing layer of the wound dressing. The airway may be arranged such that it is in fluid communication with the wound dressing.

According to a fourth aspect of the invention there is provided a negative pressure wound therapy apparatus comprising a wound dressing comprising the airway according to the first aspect of the invention (optionally including any optional features) and a source of negative pressure, for example a pump. The negative pressure wound dressing may comprise a connector connected to the distal end of the conduit and to the pump. The connector may be a luer lock. The connector may comprise a filter.

Thus, in use, the wound dressing is positioned over a wound site to form a sealed cavity over the wound site and, upon activation of the source of negative pressure, negative pressure is generated at the wound site.

According to a fifth aspect of the invention, there is provided a method of manufacturing the wound dressing according to the first aspect (optionally including any optional features). The method may comprise arranging at least part of a spacer layer in at least a first slot in a conduit. The method may comprise:
a. Forming a first slot in an end of a conduit such that the conduit comprises a first split end and a second split end; and
b. Arranging at least part of the spacer layer in the first slot.

The method may further comprise enclosing at least a portion of the spacer layer between a top layer provided over the spacer layer and a bottom layer provided below the spacer layer.

The spacer layer may comprise a proximal end, an elongate middle portion and a distal end. The proximal end of the spacer layer may be arranged in the elongate slot.

The method may further comprise forming a second slot in the end of the conduit such that the conduit comprises four split ends. The second slot may be formed perpendicular to the first slot. The spacer layer may be arranged between two pairs of split ends such that a first pair of split ends is provided above the spacer layer, in particular above a proximal end of the spacer layer, and a second pair of split ends is provided below the spacer layer, in particular below a proximal end of the spacer layer.

According to a sixth aspect of the invention, there is provided a method for the treatment of a wound, comprising:
a. Providing an airway comprising:
i. a spacer layer; and
ii. a conduit:
wherein the conduit comprises a proximal end and a distal end, and the proximal end of the conduit comprises at least a first elongate slot, wherein at least part of the spacer layer is arranged in the elongate slot,
b. Providing a source of negative pressure;
c. Attaching the conduit at its distal end to the source of negative pressure;
d. Securing the airway to a wound dressing such that the conduit is in fluid communication with the wound dressing;
e. Positioning the wound dressing over a wound site to form a sealed cavity over the wound site; and
f. Applying negative pressure to the wound site.

Negative pressure may be applied to the wound site from a pump via a connector connected to the distal end of the conduit.

The invention according to any of the first to sixth aspects may of course individually include any one or more of the features, optional or otherwise, of one another.

### DETAILED DESCRIPTION OF THE INVENTION

In order that the invention may be more clearly understood one or more embodiments thereof will now be described, by way of example only, with reference to the accompanying drawings, of which:

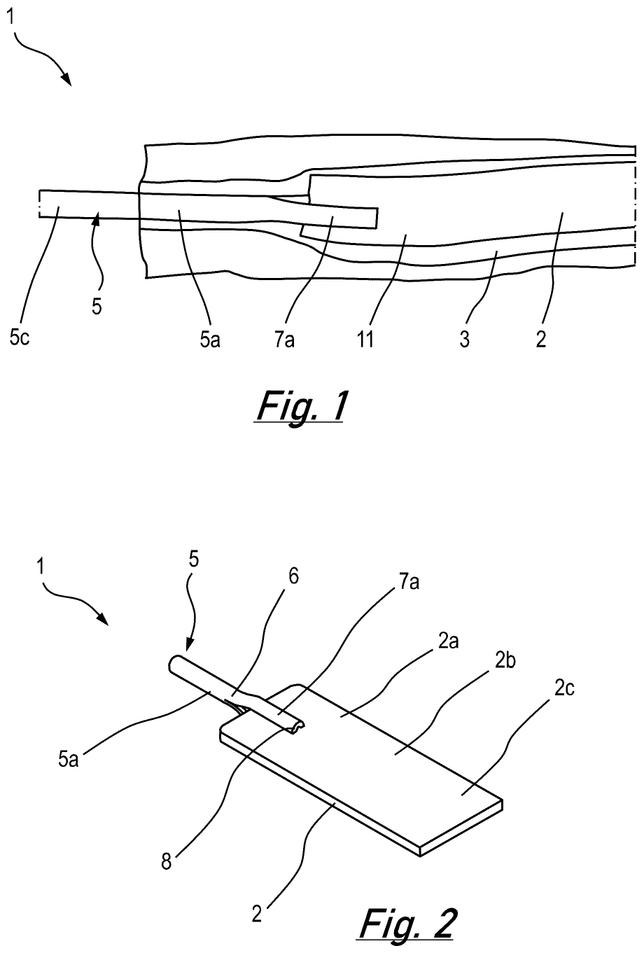

FIG. 1 is a plan view of a first embodiment of an airway;

FIG. 2 is a perspective view of part of the airway of FIG. 1;

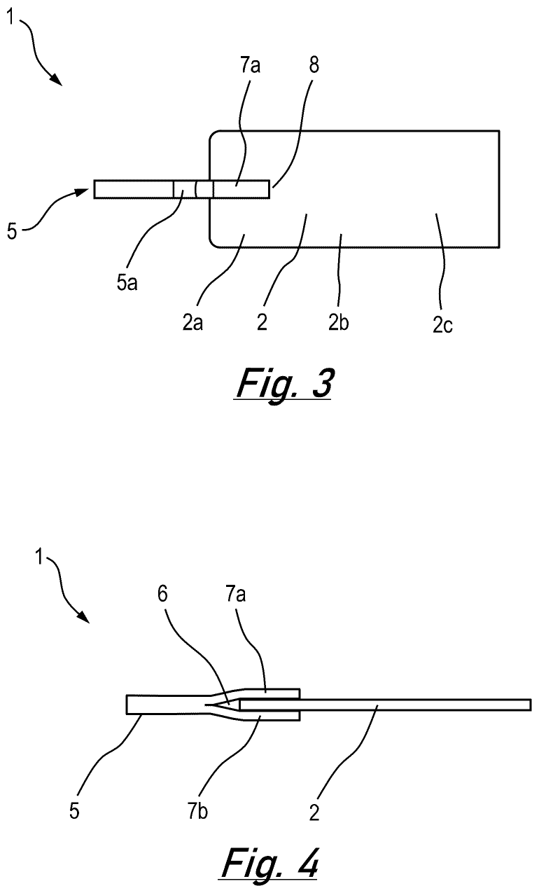

FIG. 3 is a plan view of part of the airway of FIG. 1;

FIG. 4 is a side view of part of the airway of FIG. 1;

FIG. 5 is a plan view of a second embodiment of an airway;

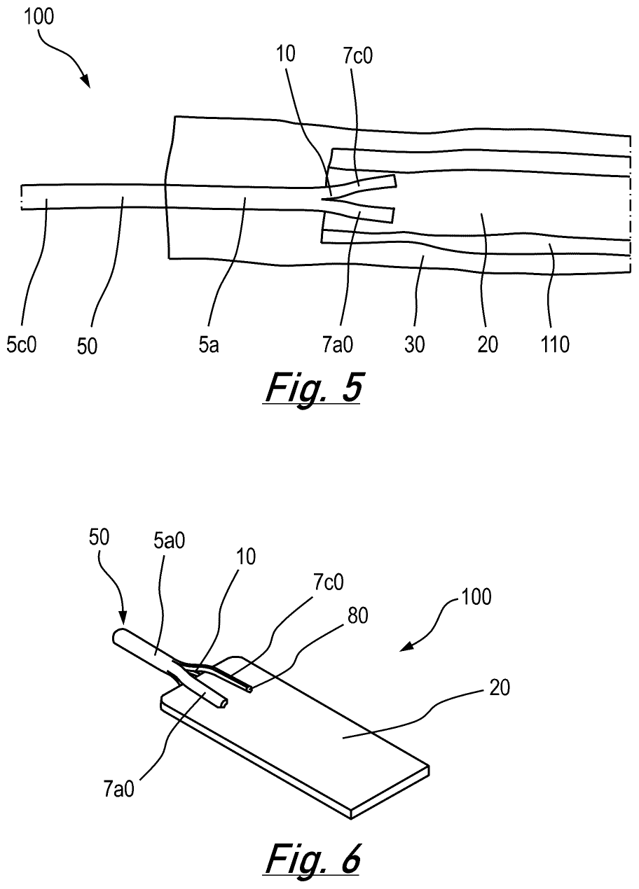

FIG. 6 is a perspective view of part of the airway of FIG. 5;

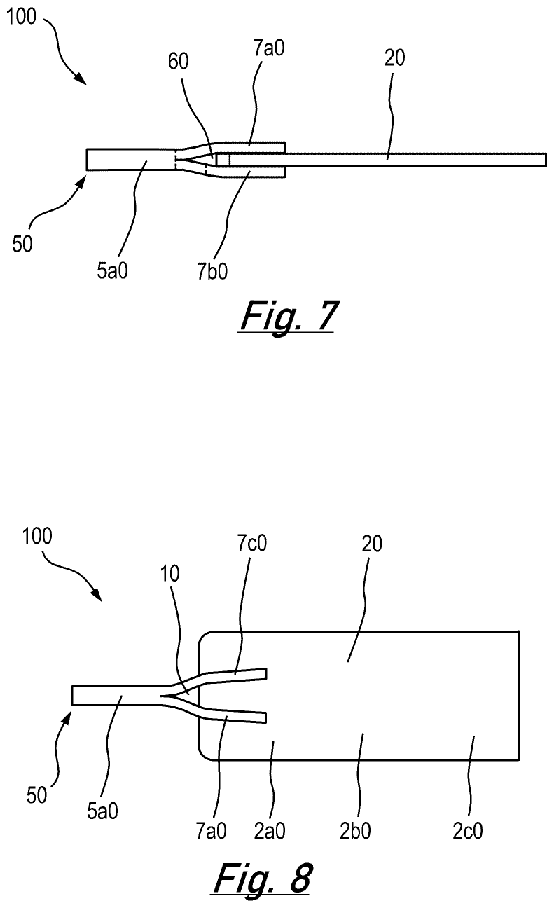

FIG. 7 is a side view of part of the airway of FIG. 5;

FIG. 8 is a plan view of part of the airway of FIG. 5;

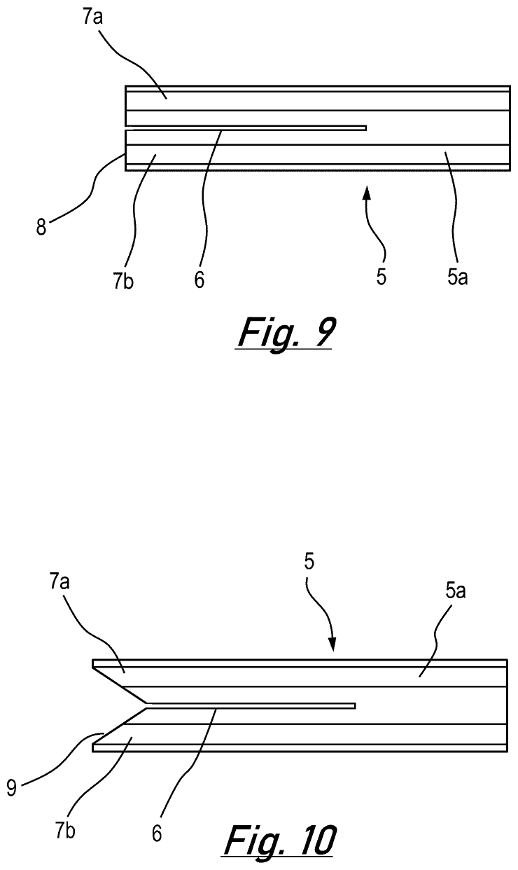

FIG. 9 is a side view of an example of a part of a conduit to be used with the airway of FIG. 1 or 5;

FIG. 10 is a side view of a further example of a part of a conduit to be used with the airway of FIG. 1 or 5;

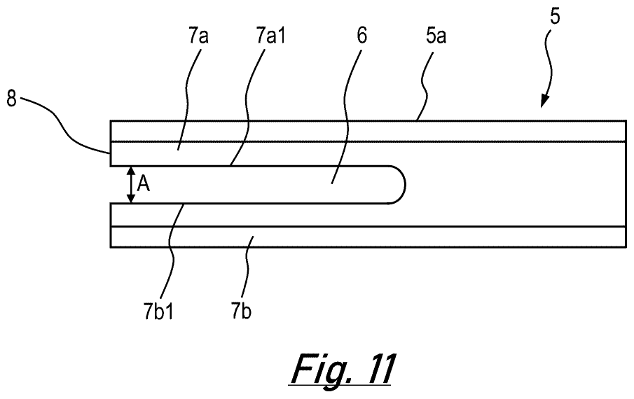

FIG. 11 is a side view of an even further example of a part of a conduit to be used with the airway of FIG. 1 or 5; and

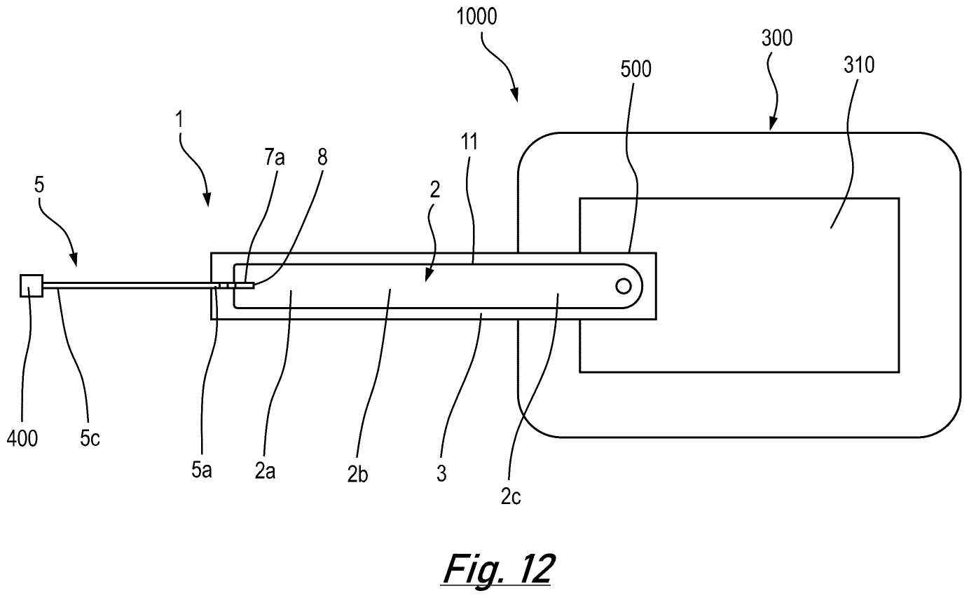

FIG. 12 is a plan view of an example of a negative pressure wound dressing comprising the airway of FIG. 1.

As used herein the expression “wound” may include an injury to living tissue may be caused by a cut, blow, or other impact, typically one in which the skin is cut or broken. A wound may be a chronic or acute injury. Acute wounds occur as a result of surgery or trauma. They move through the stages of healing within a predicted timeframe. Chronic wounds typically begin as acute wounds. The acute wound can become a chronic wound when it does not follow the healing stages resulting in a lengthened recovery. It is believed that the transition from acute to chronic wound can be due to a patient being immuno-compromised.

Chronic wounds may include for example: venous ulcers (such as those that occur in the legs), which account for the majority of chronic wounds and mostly affect the elderly, diabetic ulcers (for example, foot or ankle ulcers), peripheral arterial disease, pressure ulcers, or epidermolysis bullosa (EB).

Examples of other wounds include, but are not limited to, abdominal wounds or other large or incisional wounds (either as a result of surgery, trauma, stemiotomies, fasciotomies, or other conditions), dehisced wounds, acute wounds, chronic wounds, subacute and dehisced wounds, traumatic wounds (such as from orthopaedic trauma), flaps and skin grafts, lacerations, abrasions, contusions, burns, diabetic ulcers, pressure ulcers, stoma, surgical wounds, trauma and venous ulcers, broken bones or the like.

Wounds may also include a deep tissue injury. Deep tissue injury is a term proposed by the National Pressure Ulcer Advisory Panel (NPUAP) to describe a unique form of pressure ulcers. These ulcers have been described by clinicians for many years with terms such as purple pressure ulcers, ulcers that are likely to deteriorate and bruises on bony prominences.

The technology disclosed can be used on an acute or chronic wound.

Referring to FIGS. 1-4 and 12, an embodiment of an airway 1 is shown.

The airway 1 comprises a spacer layer 2 and a conduit 5. The conduit 5 comprise proximal end 5a and a distal end 5c. The proximal end 5a is arranged in proximity to the spacer layer 2. The proximal end 5a comprises a first slot 6. The first slot 6 is an elongate slot. The first slot 6 may be formed as a diametrical slit in the terminus of the proximal end 5a of the conduit 5. The length of the first slot 6 may be equal to 10% of the length of the conduit 5 and extends linearly into the conduit.

In this embodiment, the proximal end 5a of the conduit 5 may be formed by a straight cut 8.

The first elongate slot 6 forms a first split end 7a and a second split end 7b. The first split end 7a and the second split end 7b may each have the same length.

The spacer layer 2 comprises a proximal end 2a, elongate middle portion 2b and a distal end 2c. The spacer layer 2 may have a substantially rectangular cross-sectional dimension when viewed in plan, and the width of each of the proximal end 2a, elongate middle portion 2b and the distal end 2c of the spacer layer 2 are substantially equal. The spacer layer 2 may be substantially planar.

In this embodiment, the spacer layer 2 is a strip of polyester spacer fabric. The spacer layer may have a thickness of 1.75 mm.

To manufacture the airway, the first split end 7a and the second split end 7b are separated, for example the first split end 7a may be moved upwardly and the second split end 7b may be moved downwardly, to expose the first elongate slot 6 into which the proximal end 2a of the spacer layer 2 is arranged. Separation of the split ends 7a, 7b may be by any convenient method but may include simply pushing the proximal end 2a of the spacer layer 2 into the first slot 6 or by gently pinching the conduit 5 at the base of the first slot 6.

The first split end 7a may be provided above the proximal end 2a of the spacer layer 2. The second split end 7b may be provided below the proximal end 2a of the spacer layer 2. Each split end 7a, 7b may extend across the proximal end 2a of the spacer layer 2 (toward the elongate middle portion 2b) to a distance equal to 5% of the length of the spacer layer.

The spacer layer 2 may be held between the first split end 7a and the second split end 7b by friction. That is to say that the first split end 7a and the second split end 7b function as a clamp to grip the proximal end 2a of the spacer layer 2. In other embodiments, an adhesive may be used to adhere the splits ends 7a, 7b to the spacer layer 2.

The first split end 7a and the second split end 7b may each taper towards their respective tip. Advantageously, this means that each split end 7a, 7b comprises an outer surface which becomes flatter towards its end and, being thinner, is more flexible towards its end, and therefore does not comprise a hard/sharp end point which the top layer 3 or bottom layer 4 could be pressed against and which may, albeit unlikely, cause the conduit 5 to pierce the respective film layer 3, 4. As such, beneficially, in embodiments where each split end 7a, 7b tapers, the possibility that the conduit will pierce the top and/or bottom film layers is further reduced.

The first elongate slot 6 may be formed as a slit, with no material removed from the proximal end 5a of the conduit 5. As such, the split ends 7a, 7b may meet at their bases. In other embodiments (not shown), the first elongate slot 6 may be formed by removing material of the conduit, such that the split ends 7a, 7b are separated by a gap at their bases.

The airway 1 further comprises a top layer 3 constructed from a liquid impermeable material. In this embodiment, the top layer 3 is a film layer formed of polyurethane. The top layer 3 is provided over the spacer layer 2.

The airway 1 further comprises a bottom layer 4 constructed from a liquid impermeable material. In this embodiment, the bottom layer 4 is a film layer formed of polyurethane. The bottom layer 4 is provided below the spacer layer 2.

The top layer 3 and the bottom layer 4 are each waterproof, gas impermeable and moisture vapour permeable.

The top layer 3 and the bottom layer 4 substantially enclose the spacer layer 2 and the proximal end 5a of the conduit 5. The top layer 3 may cover the entire upper surface of the spacer layer 2 and the bottom layer 4 may cover the majority of the underside of the spacer layer. The top layer 3 and bottom layer 4 may be joined by heat lamination, but preferably an adhesive, to enclose the spacer layer 2 at the sides and, therefore, form a substantially rectangular elongate channel 11 holding the spacer layer 2 and the proximal end 5a of the conduit 5 therein, but where the airway 1 is connected to a wound dressing 300, it is not enclosed by the top layer 3 and bottom layer 4; in particular, the top layer 3 may be bonded, for example by an adhesive, to an upper surface 310 of the wound dressing 300, rather than the bottom layer 4 to enclose the spacer layer 2 in that region and provide a fluid-tight channel between the opening in the wound dressing 300 and the conduit 5. The underside of the spacer layer 2 may be unenclosed in the region 500 of an aperture in the upper surface 310 of the wound dressing 300. The aperture being in fluid communication with apertures in each layer of the wound dressing 300 and the wound site to form a fluid communication pathway. In this way there is fluid communication between interior of the wound dressing 300 and the airway 1.

In use as part of a negative pressure wound dressing 1000, the conduit 5 is attached at its distal end 5c to a pump (not shown) which functions as a source of negative pressure and is capable of supplying negative pressure, possibly via a connector 400 or an adapter. Upon activation of the pump, air is drawn toward the pump from the wound site, through the fluid communication pathway and the elongate channel 11 and along the conduit 5, and a negative pressure is therefore transmitted to the wound site.

FIGS. 5 to 8 show an airway 100 according to a second embodiment.

The airway 100 comprises a spacer layer 20 and a conduit 50. The conduit 50 comprise proximal end 5a0 and a distal end 5c0. The proximal end 5a0 is arranged in proximity to the spacer layer 20.

The proximal end 5a0 comprises a first slot 60. The first slot 60 is an elongate slot. The first slot 60 is formed as a diametrical slot in the terminus of the proximal end 5a0 of the conduit 50. The length of the first slot 60 is equal to 10% of the length of the conduit 50 and extends linearly into the conduit.

In this embodiment, the proximal end 5a0 of the conduit 50 comprises a second elongate slot 10. The second elongate slot 10 is formed as a diametrical slot in the terminus of the proximal end 5a0 of the conduit 50. The second elongate slot 10 is perpendicular to the first elongate slot 60. The length of the second slot 10 is equal to 10% of the length of the conduit 50 and extends linearly into the conduit.

In this embodiment, the proximal end 5a0 of the conduit 50 is formed by a straight cut 80.

The first elongate slot 60 forms a first split end 7a0 and a second split end 7b0. The first split end 7a0 and the second split end 760 each have the same length.

The second elongate slot forms a third split end 7c0 and a fourth split end 7d0. The third split end 7c0 is of equal dimensions to the first split end 7a0. The fourth split end 7d0 is of equal dimensions to the second split end 7b0.

The first split end 7a0 is diametrically opposed to the fourth split end 7d, and the second split end 7b0 is diametrically opposed to the third split end 7c0.

The spacer layer 20 comprises a proximal end 2a0, elongate middle portion 2b0 and a distal end 2c0. The spacer layer 20 has a substantially rectangular cross-sectional dimension when viewed in plan view, and the width of each of the proximal end 2a0, elongate middle portion 2b0 and the distal end 2c0 of the spacer layer 20 are substantially equal. The spacer layer 20 is substantially planar.

The proximal end 2a0 of the spacer layer 20 is arranged between a first pair of split ends comprising the first 7a0 and third 7c0 split ends, and a second pair of split ends comprising the second 7b0 and fourth 7d0 split ends.

The first split end 7a0 and the third split end 7c0 are provided above the proximal end 2a0 of the spacer layer 20. The second split end 760 and the fourth split end 7d0 are provided below the proximal end 2a0 of the spacer layer 20. Each split end 7a0, 7b0, 7c0 and 7d0 extends across the proximal end 2a0 of the spacer layer 20 (toward the elongate middle portion 2b0) to a distance equal to 10% of the length of the spacer layer.

In this embodiment, the spacer layer is again a strip of polyester spacer fabric.

To manufacture the airway, the first split end 7a0 and the third split end 7c0 are together separated from the second split end 7b0 and the fourth split end 7d0, for example the first split end 7a0 and the third split end 7c0 may be moved upwardly and the second split end 760 and the fourth split end 7d0 may be moved downwardly, to expose the first elongate slot 60 into which the proximal end 2a0 of the spacer layer 20 is arranged. Separation of the split ends 7a0 and 7c0 from 7b0 and 7d0 may be by any convenient method but may include simply pushing the proximal end 2a0 of the spacer layer 20 into the first slot 60 or by gently pinching the conduit 50 at the base of the first slot 60 and second slot 10, or by gently using the spacer layer 20 to separate each pair of split ends.

The spacer layer 2 is held between the first pair of split ends comprising the first split end 7a0 and the third split end 7c0 and the second pair of split ends comprising the second split end 7b0 and the fourth split end 7d0 by friction. That is to say that each pair of split ends grip the proximal end 2a0 of the spacer layer 20. The first pair of split ends comprising the first split end 7a0 and the third split end 7c0 exert a force downwardly onto an upper surface of the spacer layer 20, and the second pair of split ends comprising the second split end 7b0 and the fourth split end 7d0 exert a force upwardly onto a lower surface of the spacer layer 20.

The airway 100 further comprises a top layer 30 constructed from a liquid impermeable material. In this embodiment, the top layer 30 is formed of polyurethane. The top layer 30 is provided over the spacer layer 20.

The airway 100 further comprises a bottom layer 40 constructed from a liquid impermeable material. In this embodiment, the bottom layer 40 is formed of polyurethane. The bottom layer 40 is provided below the spacer layer 20.

The top layer 30 and the bottom layer 40 are each waterproof, gas impermeable and moisture vapour permeable.

The top layer 30 and the bottom layer 40 enclose the spacer layer 20 and the proximal end 5a0 of the conduit 50.

As shown in FIG. 8, the top layer 30 is adhered to the bottom layer 40 around its periphery to form a substantially rectangular elongate channel 110 holding the spacer layer 20 and the proximal end 5a0 of the conduit 50 therein. Adhering the bottom layer 40 to the top layer 30 about their respective peripheries prevents erroneous adhesion of the top 30 and/or bottom 40 layers to the spacer layer 20, which could affect the flow of air through the airway, therefore, preventing an efficient negative pressure being transmitted to the wound site, in use. The top layer 30 is adhered to the bottom layer 40 by heat lamination.

In use as part of a negative pressure wound dressing, the airway 100 functions in much the same way as airway 1 described above.

FIGS. 9 and 10 each show an example of part of a conduit 5.

FIG. 9 shows a proximal end 5a of a conduit 5. The conduit 5 having a straight cut 8 in the terminus of its proximal end 5a. The straight cut 8 is arranged such that each split end terminates perpendicular to the elongate slot 6. The first elongate slot 6 extends linearly into the conduit 5 from the straight cut 8. In this example, the first elongate slot 6 is formed as a slit, with no material removed from the proximal end 5a of the conduit 5. As such, the split ends 7a, 7b meet at their bases.

FIG. 10 shows proximal end 5a of a conduit 5. The conduit 5 having a notched cut 9 in the terminus of its proximal end 5a. The notched cut 9 is arranged such that each split end tapers outwardly from the centre of the conduit 5 toward the tip. The notched cut is a notched triangular cut. The first elongate slot 6 extends linearly into the conduit 5 from the notched cut 9. In this example, the first elongate slot 6 is formed as a slit, with no material removed from the proximal end 5a of the conduit 5. As such, the split ends 7a, 7b meet at their bases.

FIG. 11 shows a proximal end 5a of a conduit 5. The conduit 5 having a straight cut 8 in the terminus of its proximal end 5a. The straight cut 8 is arranged such that each split end terminates perpendicular to the elongate slot 6. The first elongate slot 6 extends linearly into the conduit 5 from the straight cut 8. In this example, the first elongate slot 6 is formed by removing material from the proximal end 5a of the conduit, such that the split ends 7a, 7b are separated by a gap at their bases. The gap may have a distance A between a lowermost part 7al of the first split end 7a and an uppermost part 7b1 of the second split end 7b of 0.5 mm.

The one or more embodiments are described above by way of example only. Many variations are possible without departing from the scope of protection afforded by the appended claims.

1. An airway for a pressure gradient wound therapy device comprising
a spacer layer comprising a proximal end, an elongate middle portion and a distal end;
a top layer constructed from a liquid impermeable material provided over the spacer layer;
a bottom layer constructed from a liquid impermeable material provided below the spacer layer, wherein the top layer and the bottom layer substantially enclose the spacer layer; and
a conduit comprising a proximal end and a distal end, wherein the proximal end of the conduit comprises at least a first elongate slot, wherein the first elongate slot forms at least a first split end and a second split end in the conduit, and further wherein at least part of the spacer layer is arranged in the elongate slot.

2. An airway according to claim 1, wherein the first split end is provided above the proximal end of the spacer layer.

3. An airway according to claim 1 or claim 2, wherein the second split end is provided below the proximal end of the spacer layer.

4. An airway according to any preceding claim, wherein each split end is tapered.

5. An airway according to any preceding claim, wherein the proximal end of the conduit is formed by a straight cut.

6. An airway according to any of claims 1 to 4, wherein the proximal end of the conduit is formed by a notched triangular cut such that each split end tapers outwardly from the centre of the conduit.

7. An airway according to any preceding claim, wherein the proximal end of the conduit comprises a second elongate slot perpendicular to the first elongate slot.

8. An airway according to claim 7, wherein the second elongate slot forms a third split end and a fourth split end.

9. An airway according to claim 8, wherein the proximal end of the spacer layer is arranged between two pairs of split ends.

10. An airway according to claim 8 or 9, wherein the proximal end of the spacer layer is arranged between a first pair of split ends comprising the first and third split ends, and a second pair of split ends comprising the second and fourth split ends.

11. An airway according to any preceding claim, wherein the first elongate slot is equal to no more than 50% of the length of the conduit.

12. An airway according to any of claims 7 to 11, wherein the second elongate slot is equal to no more than 50% of the length of the conduit.

13. An airway according to any preceding claim, wherein the spacer layer is planar.

14. An airway according to any preceding claim, wherein the spacer layer is a foam, a 3D spacer fabric, a 3D knitted material, a braided material, a porous material or a non-woven material.

15. An airway according to claim 14, wherein the 3D spacer fabric is formed of polyester.

16. An airway according to claim 14, wherein the foam is a polyurethane foam.

17. An airway according to any preceding claim, wherein each split end extends across the proximal end of the spacer layer to no more than 10% of the length of the spacer layer.

18. An airway according to any preceding claim, wherein the spacer layer is held between the first and second split ends by friction.

19. An airway according to any of claims 1-17, wherein the spacer layer is held between the first and second split ends by an adhesive or a heat weld.

20. An airway according to any preceding claim, wherein the width of each of the proximal end, elongate middle portion and distal end of the spacer layer are substantially equal.

21. An airway according to any preceding claim, wherein the spacer layer has a substantially rectangular cross-sectional dimension.

22. An airway according to any preceding claim, wherein the spacer layer is adhered to at least one of the top and bottom layers.

23. An airway according to any preceding claim, wherein the top layer and the bottom layer are formed of polyurethane.

24. An airway according to any preceding claim, wherein the top layer is adhered to the bottom layer to form an elongate channel holding the spacer layer therein.

25. An airway according to any preceding claim, wherein the at least part of the spacer layer arranged in the first elongate slot is planar.

26. Use of an airway according to any preceding claim with a ‘portless’ negative pressure wound dressing.

27. A method for manufacturing an airway for a pressure gradient wound therapy device, the method comprising:
a. Providing a spacer layer comprising a proximal end, an elongate middle portion and a distal end;
b. Providing a conduit comprising a proximal end and a distal end
c. Forming a first slot in the proximal end of the conduit such that the proximal end comprises a first split end and a second split end;
d. Arranging at least part of the spacer layer in the first slot;
e. Enclosing at least a portion of the spacer layer between a top layer constructed from a liquid impermeable material provided over the spacer layer and a bottom layer constructed from a liquid impermeable material provided below the spacer layer.

28. A method for the treatment of a wound, comprising:
a. Providing an airway comprising:
i. a spacer layer; and
ii. a conduit:
wherein the conduit comprises a proximal end and a distal end, and the proximal end of the conduit comprises at least a first elongate slot, the first elongate slot forms at least a first split end and a second split end, wherein at least part of the spacer layer is arranged in the elongate slot,
b. Providing a source of negative pressure;
c. Attaching the conduit at its distal end to the source of negative pressure;
d. Securing the airway to a wound dressing such that the conduit is in fluid communication with the wound dressing;
e. Positioning the wound dressing over a wound site to form a sealed cavity over the wound site; and
f. Applying negative pressure to the wound site.

29. The method according to claim 28, wherein negative pressure is applied from a pump through a connector connected to the distal end of the conduit.

30. A negative pressure wound dressing comprising the airway according to any of claims 1 to 25.
